(19) United States
(12) Patent Application Publication
(10) Pub. No.: US 2016/0303316 A1
(43) Pub. Date: Oct. 20, 2016

(54) INFUSION NEEDLE, INFUSION NEEDLE UNIT, AND FLUID INFUSION APPARATUS

(71) Applicant: Seiko Epson Corporation, Tokyo (JP)

(72) Inventor: Yoshihiko MOMOSE, Shiojiri (JP)

(21) Appl. No.: 15/096,526

(22) Filed: Apr. 12, 2016

(30) Foreign Application Priority Data
Apr. 14, 2015 (JP) 2015-082286

(51) Int. Cl. A61M 5/158; A61M 5/142

(52) U.S. Cl. CPC A61M 5/158; A61M 5/142; A61M 2005/1585

(57) ABSTRACT

An infusion needle indwelled within a living body and infusing a fluid into the living body, includes a first flow path that guides a puncture needle to be inserted into and pulled out from the living body and serves as a flow path of the fluid after the puncture needle is removed, and a second flow path that communicates with the first flow path along the first flow path.

### BACKGROUND

1. Technical Field

The present invention relates to an infusion needle, an infusion needle unit, and a fluid infusion apparatus.

2. Related Art

In related art, a fluid infusion apparatus including a feeding pump, a liquid feeder, and a liquid infuser is disclosed in Patent Document 1 (JP-A-2013-252286).

The apparatus described in Patent Document 1 is used by attaching the liquid infuser to a living body and indwelling an infusion needle (infusion tube) of the liquid infuser within the living body. The liquid (e.g. insulin preparation) sent by the feeding pump via the liquid feeder to the liquid infuser is continuously infused through a flow path formed in the infusion needle into the living body.

Further, in the continuous infusion therapy of the insulin preparation, a neutral aqueous solution preparation of the so-called human insulin is used, and, when the insulin solution is stirred in contact with a hydrophobic surface, the solution is denatured and aggregates are produced according to the description in Paragraph 0005 of Patent Document 2 (JP-A-2003-220129).

The infusion needle is used while indwelling within the living body as described above, and it is possible that the needle bends at e.g. the boundary between subcutaneous fat and dermis harder than the subcutaneous fat and the flow path width (the flow path section) becomes smaller and the flow path is clogged. Further, if an aggregate is produced in the bending location of the infusion needle, the flow path may be further clogged and smooth infusion of a fluid such as a chemical into the living body is harder.

### SUMMARY

An advantage of some aspects of the invention is to provide an infusion needle, an infusion needle unit, and a liquid infusion apparatus that can smoothly infuse a fluid into a living body even in the case where a flow path is clogged due to bending or the like.

An infusion needle according to an aspect of the invention is an infusion needle indwelled within a living body and infusing a fluid into the living body, including a first flow path that guides a puncture needle to be inserted into and pulled out from the living body and serves as a flow path of the fluid after the puncture needle is removed, and a second flow path that communicates with the first flow path along the first flow path.

The other features of the invention will be made clear by the description of the accompanying drawings and the specification.

### BRIEF DESCRIPTION OF THE DRAWINGS

The invention will be described with reference to the accompanying drawings, wherein like numbers reference like elements.

FIGS. 1A and 1B show one configuration example of a fluid infusion apparatus according to a first embodiment of the invention, and FIG. 1A is a perspective view and FIG. 1B is an exploded perspective view.

FIGS. 2A and 2B are sectional explanatory diagrams of an infusion needle unit, and FIG. 2A shows a state before the infusion needle unit is attached to a living body and FIG. 2B shows a state after the infusion needle unit is attached to the living body.

FIG. 3A is a view from a direction of arrow IIIA in FIG. 2, and FIG. 3B is a sectional view along line IIIB-IIIB in FIG. 3A.

FIG. 4 is a perspective view showing one configuration example of an infusion needle.

FIG. 5A is a sectional view along line VA-VA in FIG. 4, FIG. 5B is a view from a direction of arrow VB in FIG. 4, and FIG. 5C is a sectional view along line VC-VC in FIG. 4.

FIG. 6 is an explanatory diagram showing a state in which the infusion needle bends within the living body.

FIG. 7 is a sectional view in an extension direction of an infusion needle according to a modified example.

FIGS. 8A and 8B show a configuration example of an infusion needle according to a second embodiment of the invention, and FIG. 8A is a perspective view and FIG. 8B is a view from a direction of arrow VIIIB in FIG. 8A.

FIGS. 9A and 9B show a configuration example of an infusion needle according to a third embodiment of the invention, and FIG. 9A is a perspective view and FIG. 9B is a view from a direction of arrow IXB in FIG. 9A.

FIG. 10A is an enlarged view of a tip end side of an infusion needle unit according to a fourth embodiment of the invention, and FIG. 10B is a sectional view along line XB-XB in FIG. 10A.

### DESCRIPTION OF EXEMPLARY EMBODIMENTS

At least the following matters will be disclosed by the description of the specification and the accompanying drawings.

An infusion needle indwelled within a living body and infusing a fluid into the living body is disclosed. The infusion needle includes a first flow path that guides a puncture needle to be inserted into and pulled out from the living body and serves as a flow path of the fluid after the puncture needle is removed, and a second flow path that communicates with the first flow path along the first flow path is disclosed. With this configuration, even in the case where the infusion needle bends within the living body and clogging occurs in the first flow path, the fluid can be smoothly infused into the living body.

In the infusion needle, it is desirable that the second flow path includes a slit. With this configuration, even in the case where clogging occurs in the first flow path, the fluid flowing in the first flow path may flow into the slit. Further, the puncture needle may be stably held.

In the infusion needle, it is desirable that second flow path includes an auxiliary flow path having a smaller flow path width than the first flow path. With this configuration, even in the case where clogging occurs in the first flow path, the fluid flowing in the first flow path may flow into the auxiliary flow path.

In the infusion needle, it is desirable that the second flow paths are provided on both sides of a crossing line crossing an extension direction of the first flow path. With this configuration, even in the case where the infusion needle bends within the living body and the second flow path provided on one side of the crossing line is clogged, the fluid can be smoothly infused into the living body.

In the infusion needle, it is desirable that a tapered portion having an outer diameter dimension smaller toward a tip end is provided. With this configuration, pain when the infusion needle is inserted into the living body may be reduced.

In the infusion needle, it is desirable that a flow path width of the second flow path is smaller toward the tip end in the tapered portion. With this configuration, a problem that the tip end is turned up when the infusion needle is inserted into the living body may be suppressed.

In the infusion needle, it is desirable that the tapered portion does not include the second flow path. With this configuration, the problem that the tip end is turned up when the infusion needle is inserted into the living body may be further suppressed.

An infusion needle unit is disclosed. The infusion needle unit includes an infusion needle indwelled within a living body and infusing a fluid into the living body, and a puncture needle inserted into and pulled out from the living body, wherein the infusion needle is provided with a first flow path that guides the puncture needle and serves as a flow path of the fluid after the puncture needle is removed, and a second flow path that communicates with the first flow path along the first flow path is disclosed. With this configuration, even in the case where the infusion needle bends within the living body and clogging occurs in the first flow path, the fluid can be smoothly infused into the living body.

In the infusion needle unit, it is desirable that the puncture needle is solid. With this configuration, when the puncture needle is inserted into and pulled out from the living body, pain may be reduced compared to a hollow puncture needle.

A fluid infusion apparatus is disclosed. The fluid infusion apparatus includes an infusion needle unit including an infusion needle indwelled within a living body and infusing a fluid into the living body, and a puncture needle inserted into and pulled out from the living body, and a pump that transports the fluid to the infusion needle, wherein the infusion needle is provided with a first flow path that guides the puncture needle and serves as a flow path of the fluid after the puncture needle is removed, and a second flow path that communicates with the first flow path along the first flow path is disclosed. With this configuration, even in the case where the infusion needle bends within the living body and clogging occurs in the first flow path, the fluid can be smoothly infused into the living body.

### First Embodiment

### Overview of Fluid Infusion Apparatus 1

A fluid infusion apparatus 1 according to the first embodiment of the invention is used for e.g. periodic infusion of a chemical (fluid) of insulin or the like into a living body.

### Configuration of Fluid Infusion Apparatus 1

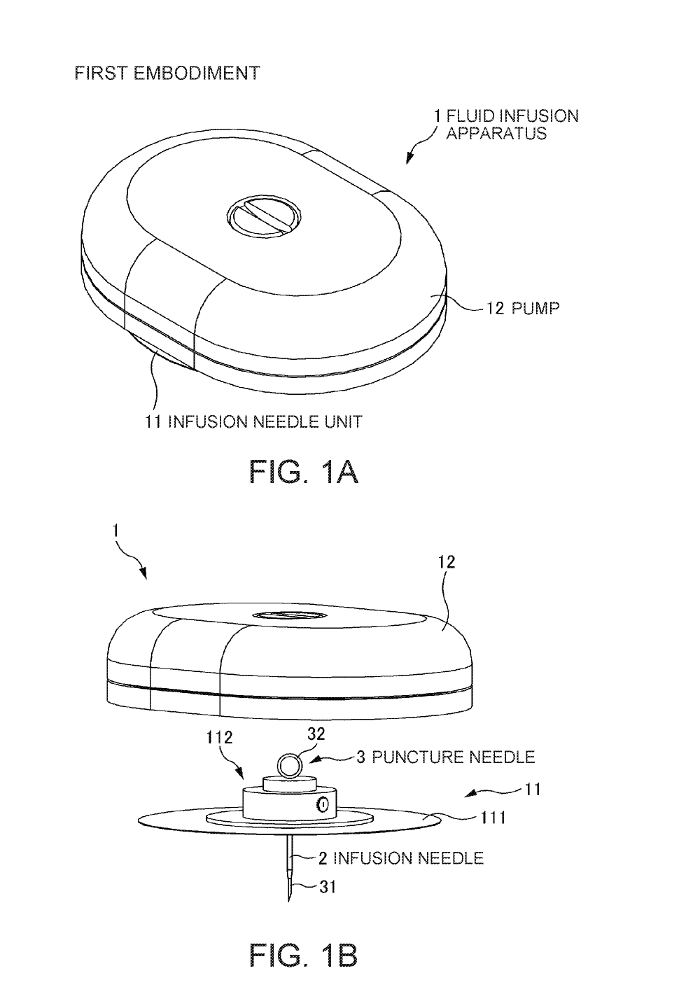

A configuration of the fluid infusion apparatus 1 will be explained with reference to FIGS. 1A and 1B.

FIGS. 1A and 1B show one configuration example of the fluid infusion apparatus 1 according to the first embodiment of the invention, and FIG. 1A is a perspective view and FIG. 1B is an exploded perspective view.

The fluid infusion apparatus 1 includes an infusion needle unit 11 having an infusion needle 2 and a puncture needle 3, and a pump 12 that infuses a fluid into the infusion needle 2. The infusion needle unit 11 and the pump 12 can be separated as shown in FIG. 1B and, for use, integrally combined as shown in FIG. 1A. Note that, in the fluid infusion apparatus 1, the infusion needle unit 11 and the pump 12 are not necessarily the separate parts, but the fluid infusion apparatus 1 having an integrated infusion needle unit 11 and pump 12 may be employed.

As shown in FIG. 1B, the infusion needle unit 11 has a seal 111 for attachment of the infusion needle unit 11 to the living body and a base 112 for attachment of the pump 12 in addition to the infusion needle 2 and the puncture needle 3.

The infusion needle 2 is a needle indwelled within the living body for infusing a fluid into the living body, and molded using e.g. fluorine resin or the like. The material of the infusion needle 2 is not necessarily the fluorine resin or the like, but may be a soft material not causing discomfort when indwelled within the living body. One end of the infusion needle 2 in the extension direction is fixed to the base 112.

The puncture needle 3 is a needle inserted into and pulled out from the living body, and molded using e.g. metal, plastic, or the like. The material of the puncture needle 3 is not necessarily metal, plastic, or the like, but may have hardness that can puncture the living body. The puncture needle 3 has a needle portion 31 at least a part of which is inserted into the living body and a tab portion 32 for pulling the puncture needle 3 from the living body.

The seal 111 has a surface facing the living body as e.g. an adhesive surface, and can be directly attached to the living body. The pump 12 has a reservoir space (not shown) for accumulation of the fluid inside.

### Use Mode of Fluid Infusion Apparatus 1

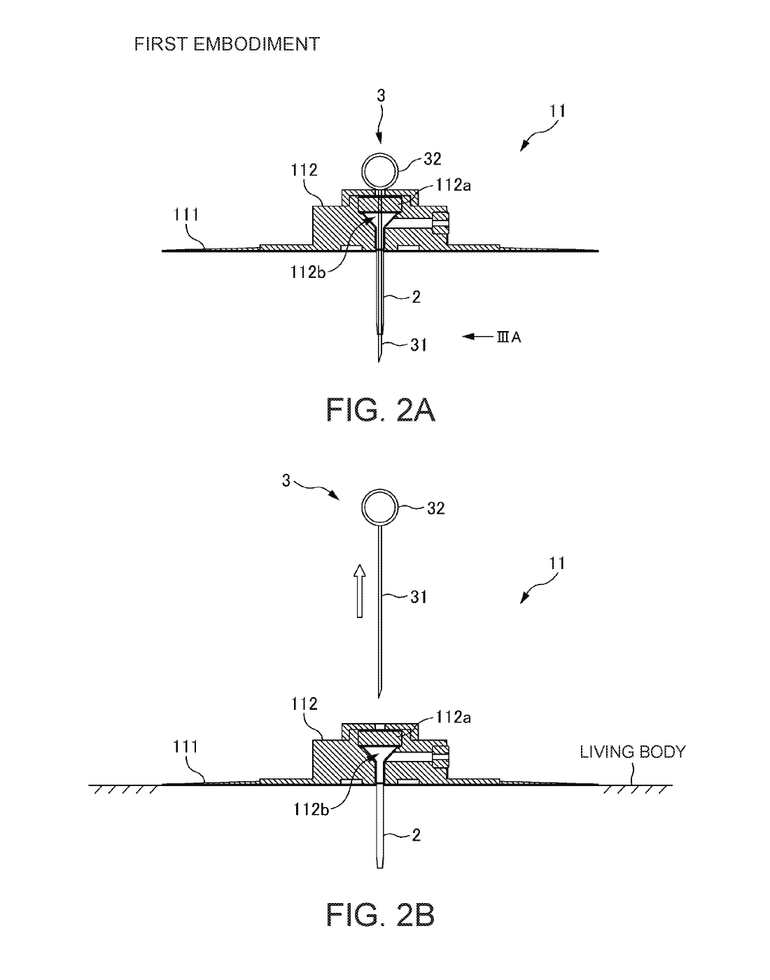

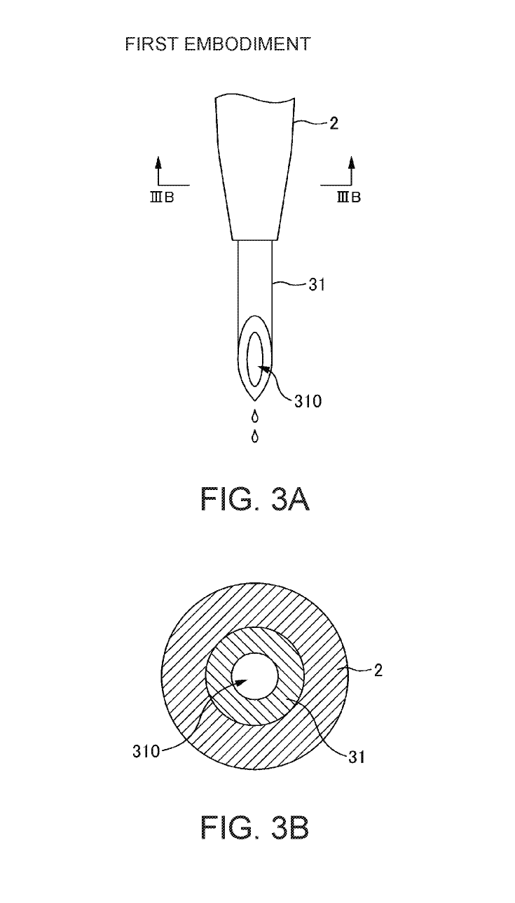

Next, a use mode of the fluid infusion apparatus 1 will be explained with reference to FIGS. 2A and 2B and FIGS. 3A and 3B.

FIGS. 2A and 2B are sectional explanatory diagrams of the infusion needle unit 11, and FIG. 2A shows a state before the infusion needle unit 11 is attached to the living body and FIG. 2B shows a state after the infusion needle unit 11 is attached to the living body. FIG. 3A is a view from a direction of arrow IIIA in FIG. 2, and FIG. 3B is a sectional view along line IIIB-IIIB in FIG. 3A.

As shown in FIG. 2A, before the infusion needle unit 11 is attached to the living body, the puncture needle 3 penetrates the infusion needle 2, the seal 11, and the base 112, and the tip end side of the needle portion 31 (the opposite side to the tab portion 32) is exposed from the infusion needle 2. In the embodiment, as shown in FIG. 3A, a hole 310 through which the fluid passes is formed along the extension direction inside of the needle portion 31. That is, as shown in FIG. 3B, the puncture needle 3 is a hollow needle. Not limited to that, but the puncture needle 3 may be a solid needle and this case will be explained in the fourth embodiment to be described later.

As shown in FIGS. 2A and 2B, within the base 112, a reservoir portion 112b for accumulating the fluid sent from the pump 12 is formed and a septum 112a molded using e.g. rubber, silicon, or the like is provided. The needle portion 31 of the puncture needle 3 has a lateral hole (not shown) in the region of the reservoir portion 112b.

When the infusion needle unit 11 is attached to the living body, a user inserts the infusion needle 2 with the puncture needle 3 into the living body. Here, in the state in which the puncture needle 3 penetrates the infusion needle 2, the fluid accumulated within the reservoir portion 112b flows into the hole 310 via the lateral hole of the needle portion 31 and passes through the hole 310 and comes out from the tip of the needle portion 31 puncturing the living body into the living body (see FIG. 3A). Thereby, the user may confirm there is no abnormality in the pump 12 or clogging before start of use in the flow path within the infusion needle unit 11 including the infusion needle 2 and the puncture needle 3. Further, after the infusion needle 2 is inserted into the living body, the fluid may be promptly infused.

Then, as shown in FIG. 2B, the user holds the tab portion 32 of the puncture needle 3 and pulls out (removes) the needle portion 31 from the living body and the infusion needle unit 11 in a direction of an arrow. Thereby, only the infusion needle 2 is dwelled within the living body. Note that, in the septum 112a, when the puncture needle 3 is pulled out, the hole formed by the puncture needle 3 is closed, and thereby, leak of the fluid within the reservoir portion 112b via the hole may be suppressed.

### Configuration of Infusion Needle 2

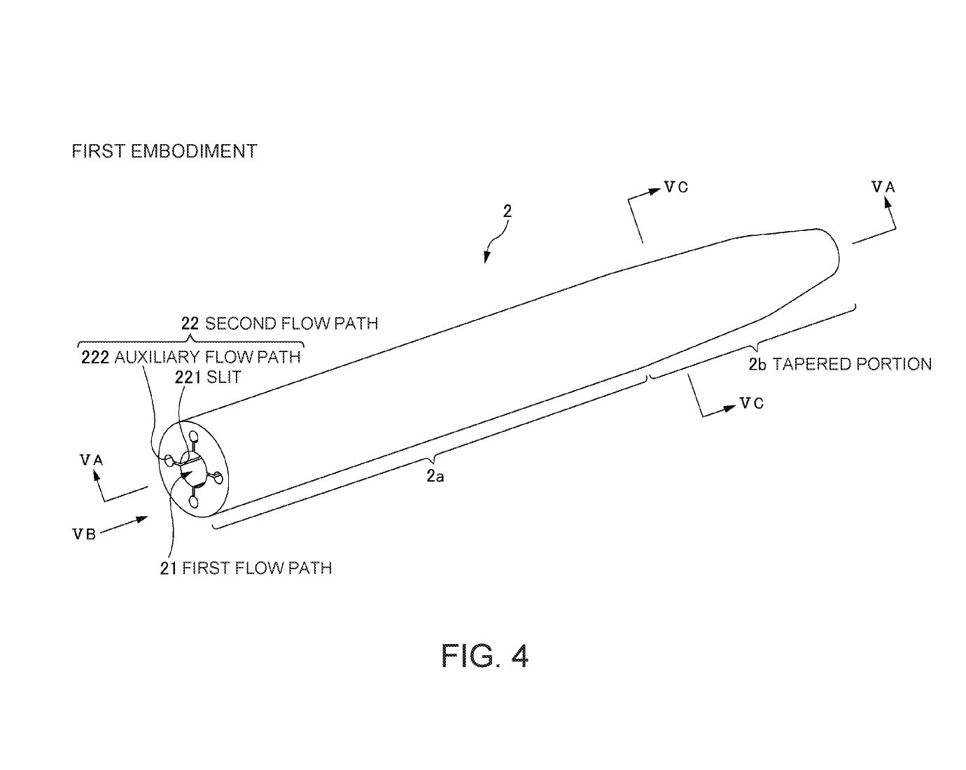

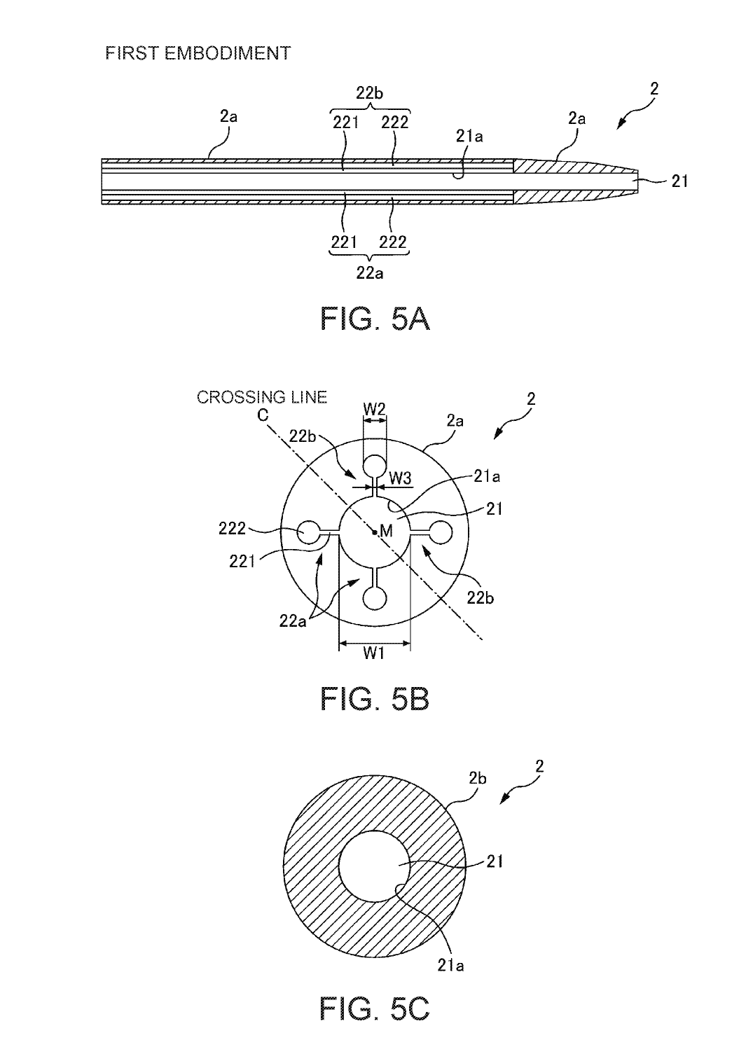

Next, a configuration of the infusion needle 2 will be explained with reference to FIGS. 4 and 5A to 5C.

FIG. 4 is a perspective view showing one configuration example of the infusion needle 2. FIG. 5A is a sectional view along line VA-VA in FIG. 4, FIG. 5B is a view from a direction of arrow VB in FIG. 4, and FIG. 5C is a sectional view along line VC-VC in FIG. 4. In FIG. 5B, a crossing line C is shown by a dashed-dotted line.

The infusion needle 2 has a first flow path 21 and second flow paths 22 inside. As the infusion needle 2, e.g. a multilumen tube in which a plurality of holes extending in the extension direction are formed may be used. The multilumen tube is molded by e.g. extrusion molding or the like.

The first flow path 21 guides the puncture needle 3 (see FIG. 1B, FIGS. 2A and 2B, and FIG. 3A) and serves as a flow path for the fluid after the puncture needle 3 is removed. The first flow path 21 penetrates the center of the infusion needle 2 in the extension direction. The fluid sent to the infusion needle 2 by the pump 12 (see FIGS. 1A and 1B) flows from one end on the base 112 side of the infusion needle 2 toward the other end (the tip puncturing the living body) within the first flow path 21 and is infused into the living body.

As shown in FIGS. 5B and 5C, the section of the first flow path 21 is a circular shape corresponding to the section shape of the puncture needle 3. Before puncturing the living body, the puncture needle 3 is held within the first flow path 21 with its outer circumference surface in contact with an inner wall 21a of the first flow path 21.

The second flow paths 22 serve as flow paths of the fluid with the first flow path 21 after removal of the puncture needle 3. The second flow paths 22 communicate with the first flow path 21 along the first flow path 21. Thereby, over entirely in the extension direction of the infusion needle 2, the fluid flowing in the first flow path 21 may flow into the second flow paths 22.

In the embodiment, as shown in FIG. 5B, in the section of the infusion needle 2, four of the second flow paths 22 are provided outside of the first flow path 21 at equal intervals of 90°. Further, regarding the second flow paths 22, the two second flow paths 22 are respectively provided on both sides of the crossing line C crossing the extension direction of the first flow path 21. More specifically, “crossing line C” refers to a line passing through a center M of the first flow path 21 and crossing the extension direction, i.e. a line crossing the center line of the first flow path 21. Hereinafter, the two second flow paths 22 provided on one side of the crossing line C are referred to as “second flow paths 22a” and the two second flow paths 22 provided on the other side of the crossing line C are referred to as “second flow paths 22b”.

In the embodiment, the second flow path 22 has a slit 221 and an auxiliary flow path 222. The slit 221 connects the first flow path 21 and the auxiliary flow path 222 over entirely in the extension direction of the infusion needle 2 and serves as a part of the flow path in which the fluid flows. The slit 221 has a nearly rectangular section and communicates with the first flow path 21 along the first flow path 21.

The auxiliary flow path 222 serves as a part of the flow path in which the fluid flows with the slit 221. The auxiliary flow path 222 has a circular section and communicates with the slit 221 along the extension direction. That is, the auxiliary flow path 222 communicates with the first flow path 21 via the slit 221. Thereby, in the infusion needle 2, the flow paths in which the fluid flows may be further secured by providing the slits 221 and the auxiliary flow paths 222 in addition to the first flow path 21.

As shown in FIG. 5B, the auxiliary flow path 222 has a flow path width W2 smaller than a flow path width W1 of the first flow path 21 (W2<W1). Further, in comparison between the flow path width W2 of the auxiliary flow path 222 and a flow path width W3 of the slit 221, the flow path width W3 of the slit 221 is formed to be smaller than the flow path width W2 of the auxiliary flow path 222 (W3<W2). Therefore, a relation among the flow path widths of the first flow path 21, the slit 221, and the auxiliary flow path 222 is such that the widths are smaller in the order of the first flow path 21, the auxiliary flow path 222, and the slit 221 (W3<W2<W1). Note that a relation among section areas of the first flow path 21, the slit 221, and the auxiliary flow path 222 is such that the areas are smaller in the order of the first flow path 21, the auxiliary flow path 222, and the slit 221 like the relation among the flow path widths.

In the infusion needle 2, the narrower slit 221 than the auxiliary flow path 222 communicates with the first flow path 21, and thereby, the area of the inner wall 21a in contact with the puncture needle 3 in the first flow path 21 may be secured to be larger, and the puncture needle 3 can be stably held.

As shown in FIGS. 4 and 5A, in the embodiment, the infusion needle 2 has a base portion 2a extending in a constant outer diameter dimension, and a tapered portion 2b in an outer diameter dimension smaller than the outer diameter dimension of the base portion 2a toward the tip puncturing the living body. Note that, in the embodiment, the tapered portion 2b changes in (tapered) ratio of reduction of the outer diameter dimension in the middle, but not limited to that. For example, the outer diameter dimension may be continuously smaller with a fixed tapered ratio. Further, the infusion needle 2 is not necessarily formed by the combination of the base portion 2a and the tapered portion 2b, but the outer diameter dimension may be continuously and gradually smaller toward e.g. the tip puncturing the living body.

Furthermore, the infusion needle 2 does not necessarily have the part with the smaller outer diameter dimension, but the outer diameter dimension may be constant over entirely in the extension direction. In this regard, one configuration example will be explained in the fourth embodiment to be described later. Note that it is desirable that the infusion needle 2 has the tapered portion 2b on the tip puncturing the living body. The tapered portion 2b is provided, and thereby, resistance between the skin and itself when the infusion needle 2 is inserted into the living body is smaller, the infusion needle 2 is easily inserted into the living body, and pain at puncture can be reduced.

As shown in FIG. 5A, the tapered portion 2b does not have the second flow paths 22, but has the first flow path 21 only. Thereby, the thickness in the tapered portion 2b may be secured and a problem that the tip end is turned up when the infusion needle 2 is inserted into the living body may be suppressed. Note that the tapered portion 2b does not necessarily have the configuration without the second flow paths 22. In this regard, one configuration example will be explained in a modified example to be described later.

### Regarding State of Bending of Infusion Needle 2

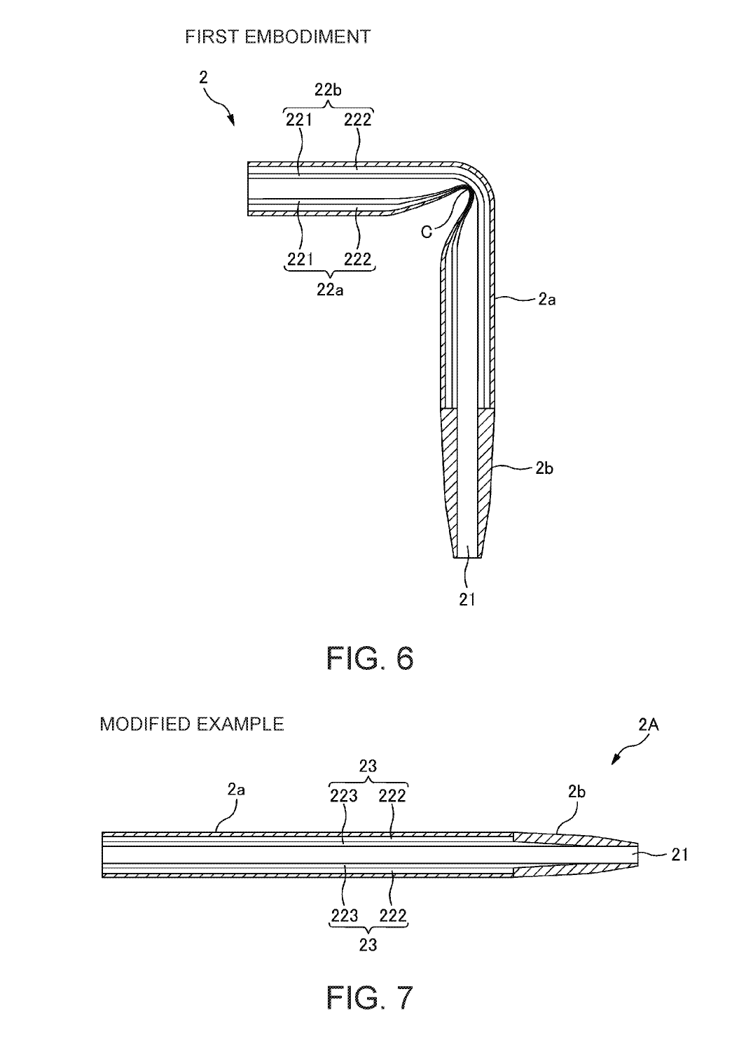

Next, a state of bending of the infusion needle 2 within the living body will be explained with reference to FIG. 6.

FIG. 6 is an explanatory diagram showing a state in which the infusion needle 2 bends on the crossing line C within the living body.

When the infusion needle 2 bends within the living body, the first flow path 21 is collapsed and clogging occurs. When the above described aggregate is produced in the bending location of the infusion needle 2, the first flow path 21 may be further clogged.

However, the infusion needle 2 has the second flow paths 22, and thereby, the fluid flowing within the first flow path 21 may flow into the second flow paths 22. In the embodiment, the fluid flowing within the first flow path 21 flows into the slits 221 and the auxiliary flow paths 222. As described above, the slits 221 and the auxiliary flow paths 222 serve as the part of the flow path, and, even when the first flow path 21 is clogged, the fluid can be smoothly infused into the living body.

As shown in FIG. 6, for example, when the infusion needle 2 bends on the crossing line C shown in FIG. 5B, in other words, when the crossing line C is a bending line, the second flow paths 22a and the first flow path 21 provided inside of the bending line are collapsed and the flow paths are closed. However, the second flow paths 22b are provided outside of the bending line, and the fluid may flow into the second flow paths 22b provided outside of the bending line. Note that FIG. 6 respectively shows only one second flow path 22a of the two second flow paths 22a provided on one side of the crossing line C and only one second flow path 22b of the two second flow paths 22b provided on the other side of the crossing line C.

The second flow paths 22 are provided outside of the crossing line C as the bending line of the infusion needle 2 and an event that all of the second flow paths 22 are collapsed depending on the position of the bending line does not occur, and thereby, the fluid may be infused into the living body more smoothly. Further, the second flow paths 22 are provided at intervals of angles less than 180° (90° in the embodiment), and thereby, the second flow paths 22 can be provided on both sides of the crossing line C (bending line).

### Modified Example

Next, a configuration of a infusion needle 2A according to a modified example of the invention will be explained with reference to FIG. 7.

FIG. 7 is a sectional view in an extension direction of the infusion needle 2A according to the modified example. In FIG. 7, the component elements in common with the infusion needle 2 according to the first embodiment have the same signs and their explanation will be omitted. As below, this applies to the second, the third, and the fourth embodiments.

The infusion needle 2A according to the modified example has a first flow path 21 and second flow paths 23 inside like the infusion needle 2 according to the first embodiment, and includes a base portion 2a and a tapered portion 2b. Note that the configurations of the second flow paths 23 are different from the configurations of the second flow path 22 according to the first embodiment. Specifically, in the second flow path 23, a slit 223 has a depth gradually shallower toward the tip end side (the opposite side to the base portion 2a) in the tapered portion 2b and the slit 223 is not provided on the tip end side of the tapered portion 2b.

Also, in this case, the thickness may be secured on the tip end side of the tapered portion 2b, and a problem that the tip end is turned up when the infusion needle 2A is inserted into the living body may be suppressed. In the infusion needle 2 according to the first embodiment, the thickness may be secured in the whole tapered portion 2b, and the problem that the tip end is turned up due to the impact when the infusion needle 2 is inserted into the living body may be further suppressed compared to the case of the modified example.

Note that, in the modified example, in the tapered portion 2b, the depth of the slit 223 is shallower toward the tip end side, however, not limited to that. For example, the flow path width of the auxiliary flow path 222 may be smaller. That is, it is only necessary that, in the tapered portion 2b, the flow path width of the second flow path 23 is smaller toward the tip end side.

### Second Embodiment

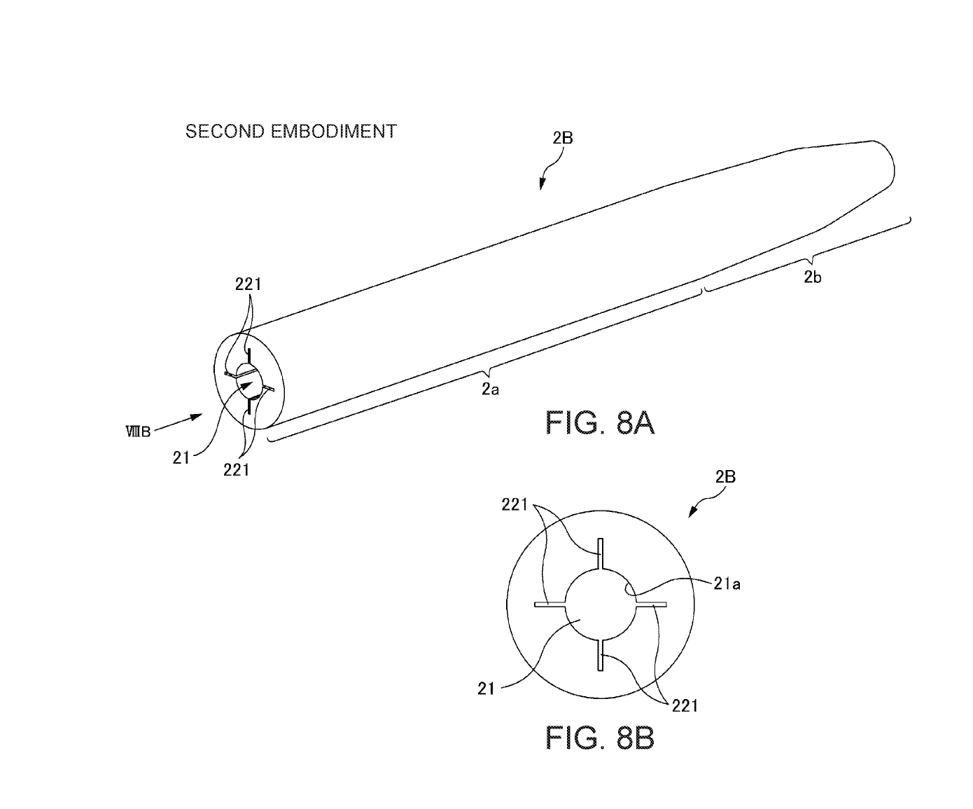

Next, a configuration of an infusion needle 2B according to the second embodiment of the invention will be explained with reference to FIGS. 8A and 8B. The infusion needle 2B according to the embodiment can be applied to the fluid infusion apparatus 1 and the infusion needle unit 11 according to the first embodiment.

FIGS. 8A and 8B show a configuration example of the infusion needle 2B according to the second embodiment of the invention, and FIG. 8A is a perspective view and FIG. 8B is a view from a direction of arrow VIIIB in FIG. 8A.

In the infusion needle 2B according to the embodiment, configurations of second flow paths are different from the configurations of the second flow paths 22 of the infusion needle 2 according to the first embodiment. Specifically, the second flow path of the infusion needle 2B includes only a slit 221 communicating with a first flow path 21 along the first flow path 21. That is, the infusion needle 2B has no auxiliary flow path. In the embodiment, as shown in FIG. 8B, four of the slits 221 are provided at equal intervals outside of the first flow path 21 in the section of the infusion needle 2B.

Also, in this case, the slits 221 serve as a part of the flow path of the fluid, and, even when clogging occurs in the first flow path 21 due to bending of the infusion needle 2B within the living body or the like, the fluid flowing within the first flow path 21 may flow into the slits 221 and the fluid may be smoothly infused into the living body. Further, the slits 221 have the narrower widths and an area of an inner wall 21a in contact with a puncture needle 3 may be secured to be larger in the first flow path 21, and the puncture needle 3 can be stably held in the first flow path 21.

### Third Embodiment

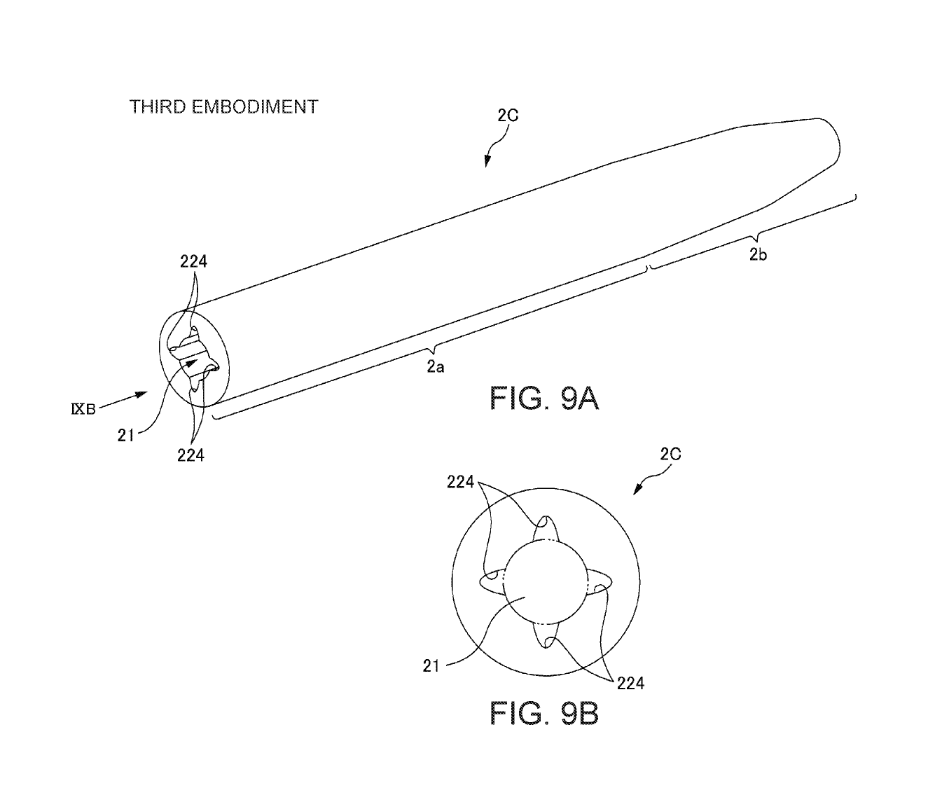

Next, a configuration of an infusion needle 2C according to the third embodiment of the invention will be explained with reference to FIGS. 9A and 9B. The infusion needle 2C according to the embodiment can be applied to the fluid infusion apparatus 1 and the infusion needle unit 11 according to the first embodiment like the infusion needle 2B according to the second embodiment.

FIGS. 9A and 9B show a configuration example of the infusion needle 2C according to the third embodiment of the invention, and FIG. 9A is a perspective view and FIG. 9B is a view from a direction of arrow IXB in FIG. 9A.

In the infusion needle 2C according to the embodiment, configurations of second flow paths are different from the configurations of the second flow paths 22 of the infusion needle 2 according to the first embodiment and the configurations of the second flow paths of the infusion needle 2A according to the second embodiment. Specifically, the second flow path of the infusion needle 2C includes only an auxiliary flow path 224 communicating with a first flow path 21 along the first flow path 21. That is, the infusion needle 2C has no slit. In the embodiment, as shown in FIG. 9B, the auxiliary flow path 224 has a nearly triangular section. Further, four of the auxiliary flow paths 224 are provided at equal intervals outside of the first flow path 21 in the section of the infusion needle 2C. Note that FIG. 9B shows a boundary between the first flow path 21 and the auxiliary flow paths 224 by a dashed two-dotted line.

Also, in this case, the auxiliary flow paths 224 serve as a part of the flow path of the fluid, and, even when clogging occurs in the first flow path 21 due to bending of the infusion needle 2B within the living body or the like, the fluid flowing within the first flow path 21 may flow into the auxiliary flow paths 224 and the fluid may be smoothly infused into the living body.

### Fourth Embodiment

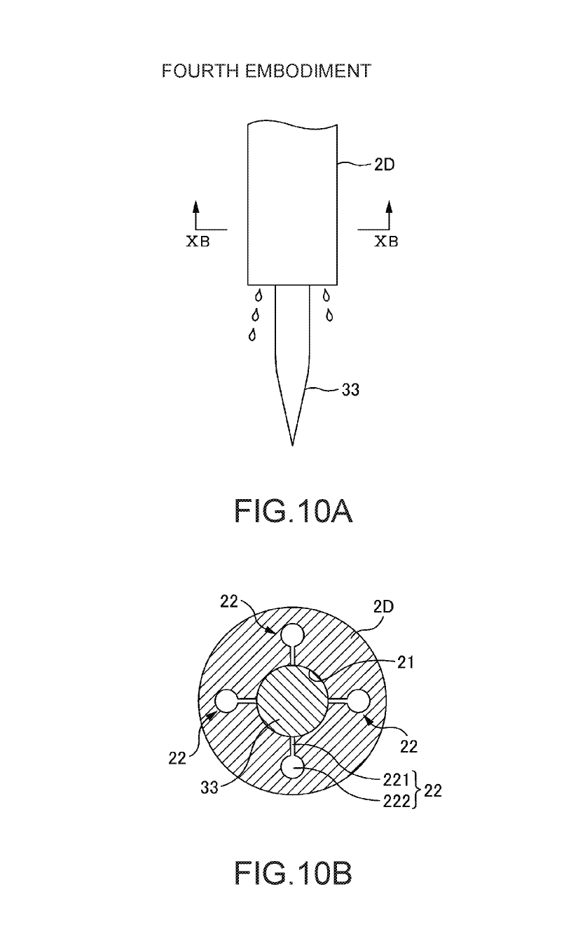

Next, a configuration of an infusion needle 2D according to the fourth embodiment of the invention will be explained with reference to FIGS. 10A and 10B. The infusion needle 2D according to the embodiment can be applied to the fluid infusion apparatus 1 and the infusion needle unit 11 according to the first embodiment like the infusion needle 2B according to the second embodiment and the infusion needle 2C according to the third embodiment.

FIG. 10A is an enlarged view of a tip end side of the infusion needle unit 2D according to the fourth embodiment of the invention, and FIG. 10B is a sectional view along line XB-XB in FIG. 10A.

The infusion needle 2D according to the embodiment has no tapered portion 2b unlike the infusion needle 2 according to the first embodiment, the infusion needle 2A according to the modified example, the infusion needle 2B according to the second embodiment, or the infusion needle 2C according to the third embodiment. That is, the infusion needle 2D is formed in a constant outer diameter dimension from one end (a base 112 side) to the other end (a side puncturing the living body) in an extension direction.

The infusion needle 2D has a first flow path 21 and second flow paths 22 inside like the infusion needle 2 according to the first embodiment. The second flow path 22 including a slit 221 and an auxiliary flow path 222 is formed from one end to the other end in the extension direction of the infusion needle 2D. That is, the infusion needle 2D also has the second flow paths 22 (slits 221 and auxiliary flow paths 222) on the tip end side puncturing the living body. Note that the second flow path 22 of the infusion needle 2D may include only the slit 221 like the infusion needle 2B according to the second embodiment, or may include only the auxiliary flow path 222 like the infusion needle 2C according to the third embodiment.

As shown in FIG. 10A, a puncture needle 33 is a solid needle having no hole formed to extend in the extension direction inside unlike the puncture needle 3 explained in the first embodiment. When the infusion needle 2D and the puncture needle 33 are inserted into the living body, the puncture needle 33 is held within the first flow path 21, and thereby, as shown in FIG. 10B, the first flow path 21 is closed by the puncture needle 33 and, when the puncture needle 33 is removed, no fluid exists within the first flow path 21. Thus, if the infusion needle 2D has no second flow path 22, a problem that it is impossible for the user to confirm the fluid coming out into the living body and a problem that it is hard to promptly infuse the fluid when the puncture needle 33 is removed arise.

However, in the embodiment, the infusion needle 2D has the second flow paths 22 to the tip puncturing the living body, and thereby, the fluid can flow into the living body via the second flow paths 22 and, when the puncture needle 33 is removed, a liquid exists within the second flow paths 22. Therefore, even when the solid puncture needle 33 is used, the user may confirm the fluid coming out into the living body (see FIG. 10A) and the fluid can be promptly infused when the puncture needle 33 is removed. Further, the puncture needle 33 is solid, and thereby, compared to the hollow puncture needle, pain when inserted into the living body may be made smaller, the strength of the puncture needle 33 may be made higher, and the infusion needle 2 and the puncture needle 33 may be molded to be thinner. Furthermore, the solid puncture needle 33 may be manufactured at a lower price than the hollow puncture needle and the cost may be reduced.

### Additional Matters

The above described embodiments are for facilitation of understanding of the invention, not for limited interpretation of the invention. Changes, improvements may be made to the invention without departing from the scope thereof, and, obviously, the invention includes their equivalents.

The entire disclosure of Japanese Patent Application No. 2015-082286 filed Apr. 14, 2015 is expressly incorporated by reference herein.

What is claimed is:

1. An infusion needle indwelled within a living body and infusing a fluid into the living body, comprising:
a first flow path that guides a puncture needle to be inserted into and pulled out from the living body and serves as a flow path of the fluid after the puncture needle is removed; and
a second flow path that communicates with the first flow path along the first flow path.

2. The infusion needle according to claim 1, wherein the second flow path includes a slit.

3. The infusion needle according to claim 1, wherein the second flow path includes an auxiliary flow path having a smaller flow path width than the first flow path.

4. The infusion needle according to claim 1, wherein the second flow paths are provided on both sides of a crossing line crossing an extension direction of the first flow path.

5. The infusion needle according to claim 1, further comprising a tapered portion having an outer diameter dimension smaller toward a tip end.

6. The infusion needle according to claim 5, wherein a flow path width of the second flow path is smaller toward the tip end in the tapered portion.

7. The infusion needle according to claim 5, wherein the tapered portion does not include the second flow path.

8. An infusion needle unit comprising:
an infusion needle indwelled within a living body and infusing a fluid into the living body; and
a puncture needle inserted into and pulled out from the living body,
wherein the infusion needle is provided with a first flow path that guides the puncture needle and serves as a flow path of the fluid after the puncture needle is removed, and a second flow path that communicates with the first flow path along the first flow path.

9. The infusion needle according to claim 8, wherein the puncture needle is solid.

10. A fluid infusion apparatus comprising:
an infusion needle unit including an infusion needle indwelled within a living body and infusing a fluid into the living body, and a puncture needle inserted into and pulled out from the living body; and
a pump that transports the fluid to the infusion needle,
wherein the infusion needle is provided with a first flow path that guides the puncture needle and serves as a flow path of the fluid after the puncture needle is removed, and a second flow path that communicates with the first flow path along the first flow path.
